(19) United States
(12) Patent Application Publication
(10) Pub. No.: US 2006/0286047 A1
(43) Pub. Date: Dec. 21, 2006

(54) Methods for determining the sequence of a peptide motif having affinity for a substrate

(76) Inventor: David J. Lowe, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/157,661

(22) Filed: Jun. 21, 2005

(51) Int. Cl. C40B 30/02; G01N 33/53; G06F 19/00; A61K 8/65

(52) U.S. Cl. 424/61; 702/19; 424/70.14; 435/7.1

(57) ABSTRACT

Disclosed herein are methods for determining peptide motifs having binding affinity for a specified substrate. The method proceeds through the analysis of a population of peptides having some affinity for a substrate for the identification of the presence of subsequences that occur statistically more frequently than by random chance. These subsequences are then assembled into motifs having reproducible strong binding affinity for the subject substrate.

### FIELD OF THE INVENTION

The invention relates to the field of data analysis. More specifically, the invention relates to methods for identifying peptide motifs having affinity for a particular substrate.

### BACKGROUND OF THE INVENTION

Since its introduction in 1985, phage display has been widely used to discover a variety of ligands including peptides, proteins and small molecules for drug targets. The applications have expanded to other areas such as studying protein folding, novel catalytic activities, DNA-binding proteins with novel specificities, and novel peptide-based biomaterial scaffolds for tissue engineering.

More recently, phage display has been used to identify peptide sequences that have a binding affinity for a particular substrate. For example, Whaley et al. (Nature 405:665-668 (2000)) disclose the use of phage display screening to identify peptide sequences that can bind specifically to different crystallographic forms of inorganic semiconductor substrates. Jagota et al. (copending and commonly owned U.S. patent application Ser. No.10/453415 and WO 03102020) describe the use of phage display to identify carbon nanotube-binding peptides. Phage display has also been used to identify peptides that bind to hair, skin, and nails (Estell et al. WO 0179479; Murray et al., U.S. Patent Application Publication No. 2002/0098524; Janssen et al., U.S. Patent Application Publication No. 2003/0152976; Janssen et al., WO 04048399; and Huang et al., copending and commonly owned U.S. patent application Ser. No.10/935642 and U.S. Patent Application Publication No. 2005/0050656) for use in personal care compositions, and to pigments and print media (O'Brien et al., copending and commonly owned U.S. patent application Ser. No. 10/935254 and U.S. Patent Application Publication No. 2005/0054752) for use in dispersants for printing and coating applications.

Pattern recognition is a well-established discipline in computer science that can be used to identify peptide binding motifs from data generated from phage display and other combinatorial methods. For example, Waterman et al. (Bulletin of Mathematical Biology 46:512-527 (1984)) describe a method for comparing several sequences in order to find consensus patterns that occur imperfectly above a preset frequency. Myers et al. (Comput. Appl. Biosci. 9:299-314 (1993)) describe a system called ANREP for finding matches to patterns composed of spacing constraints called spacers and approximate matches to motifs. Vaidyanathan et al. (copending and commonly owned U.S. patent application Ser. No. 09/851674, and U.S. Patent Application Publication No. 2003/0220771) describe a method of discovering one or more patterns in two sequences of symbols that involves the formation of a master offset table for each sequence, which groups the position for each symbol in the sequence occupied by each occurrence of that symbol. These methods are very useful for identifying peptide motifs from data generated from phage display and other combinatorial methods.

However, phage display, as typically practiced, requires many rounds of biopanning to give a few peptide sequences with strong binding properties. Successive rounds of biopanning may reduce signals in the data more than background, so that some binding sequences may not be identified. Additionally, phage display can yield peptide sequences wherein only a part of the sequence binds specifically to the substrate. Moreover, phage display is unlikely to identify long peptide sequences wherein all the amino acid residues participate in binding because the library contains only a small fraction of all possible sequences and shorter subsequences that are far more abundant occupy the binding sites on the substrate.

Therefore, the need exists for a data analysis method that can be used to determine peptide binding motifs from data obtained from phage display or other combinatorial methods wherein only a few rounds of biopanning are used. The method should be capable of generating long peptide sequences wherein all of the amino acid residues participate in binding.

Applicants have addressed the stated need by discovering a data analysis method for non-empirically determining peptide motifs having affinity for a particular substrate. The method involves an analysis of a population of peptides that have been determined to have substrate binding characteristics. The population of substrate binding peptides is further analyzed to identify frequently occurring subsequences that are then assembled into motifs with substrate binding properties.

### SUMMARY OF THE INVENTION

The invention provides methods for non-empirically determining and generating the sequence of peptide motifs that have particular binding affinity for certain substrates, such as body surfaces, pigments, print media, carbon nanotubes, semiconductors, and various polymers. The method advances the art where, previously determination of peptides having specific binding affinities has relied on various screening and bio-panning methods.

Accordingly, the invention provides a method for non-empirically generating a sequence of a peptide motif having binding affinity for a substrate comprising the steps of:
a) providing a first population of substrate-binding peptides, each having a known amino acid sequence;
b) identifying all subsequences comprising at least two amino acids contained within the population of substrate-binding peptides of (a);
c) selecting those subsequences of (b) that occur statistically more frequently than by random chance to produce a statistically significant population of subsequences;
d) identifying multiples of statistically significant subsequences that have at least two amino acid patterns in common; and
e) assembling the multiples of statistically significant subsequences of (d) to generate at least one new peptide motif having binding affinity for a substrate, wherein said new peptide motif is not contained within the first population of substrate-binding peptides.

In another embodiment the invention also provides peptide motifs having binding affinity for hair, and hair binding compositions comprising these peptide motifs.

In an additional embodiment the invention provides methods for modifying hair using the hair binding compositions of the invention.

In another embodiment the invention provides hair care, skin care, tooth care and nail care compositions comprising peptide motifs generated by the non-empirical methods of the invention.

In additional embodiments the invention provides specific peptide motif having binding affinity for hair selected from the group consisting of: SEQ ID NOs:81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 20, 121, 122, and 123.

### BRIEF DESCRIPTION OF SEQUENCE DESCRIPTIONS

The invention can be more fully understood from the following detailed description and the accompanying sequence descriptions, which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-80 are the amino acid sequences of members of a population of bleached hair-binding peptides identified by phage display screening.

SEQ ID NO:81-123 are the amino acid sequences of the generated hair-binding peptide motifs of the invention.

SEQ ID NO:124 is the amino acid sequence of a control hair-binding peptide used in Example 4.

SEQ ID NO: 125 is the amino acid sequence of the Caspase 3 cleavage site.

SEQ ID NO:126 is the oligonucleotide primer used to sequence phage DNA.

SEQ ID NOs:127 and 128 are the amino acid sequences of the reference subsequences used in Example 2

### DETAILED DESCRIPTION OF THE INVENTION

The present invention relates to non-empirical methods of determining and generating the sequence of peptide motifs that have particular binding affinity for certain substrates. Substrates of particular interest are those of importance in the personal care industry, including but not limited to, body surfaces, such as hair, skin, nails, teeth, surfaces of the oral cavity, and the like. The method may also be used to identify peptide motifs that have particular binding affinity for other substrates, such as pigments, print media, carbon nanotubes, semiconductors, and various polymers. The method is non-empirical and involves an analysis of a population of peptides that have been determined to have substrate binding characteristics. The population of substrate binding peptides is then further analyzed to identify frequently occurring subsequences that are then assembled into motifs with substrate binding properties.

The invention is useful for rapidly identifying peptides that strongly bind to commercially useful substrates from a data set of peptides that have some binding affinity for the substrate. The invention advances the art by greatly reducing the cycle time required for the identification of peptides with useful binding characteristics opposite standard biopanning methods. The resultant peptides have utility in many compositions, useful in the personal care, printing, and electronics industries.

The following definitions and abbreviations are to be used for the interpretation of the claims and the specification.

The term “non-empirical” as used in the context of generating or selecting peptide motifs means an analytical method that does not rely completely on physical selection processes such as activity screening of peptides or biopanning.

The term “peptide motif” as used herein, refers to a peptide sequence having a binding affinity for a particular substrate.

The term “peptide” refers to two or more amino acids joined to each other by peptide bonds or modified peptide bonds.

The term “binding affinity” refers to the ability of a peptide motif to interact (i.e., associate) with its respective substrate. The strength of the interaction may be determined using methods known in the art, for example an enzyme-linked immunoassay (ELISA)-based binding assay or a radiochemical binding assay.

The phrase “population of substrate-binding peptides” refers to a group of peptide sequences that have been identified using combinatorial methods to have some binding affinity for a particular substrate.

The term “substrate” refers to a material or substance for which it is desired to identify specific peptide sequences that bind thereto. Examples of substrates include, but are not limited to, body surfaces, pigments, print media, carbon nanotubes, semiconductors, and polymers.

The term “body surface” refers to any surface of the human body that may serve as a substrate for the binding of a peptide carrying a benefit agent. Typical body surfaces include, but are not limited to, hair, skin, nails, teeth, gums, surfaces of the oral cavity, and corneal tissue.

The term “benefit agent” is a general term applying to a compound or substance that may be coupled with a binding peptide for application to a body surface. Benefit agents typically include conditioners, colorants, fragrances, whiteners and the like, along with other substances commonly used in the personal care industry.

The term “hair” as used herein refers to human hair, eyebrows, and eyelashes.

The term “skin” as used herein refers to human skin, or pig skin, or substitutes for human skin such as Vitro-Skin® and EpiDerm™.

The term “nails” as used herein refers to human fingernails and toenails.

The term “carbon nanotube” refers to a hollow article comprised primarily of carbon atoms, however the nanotube may be doped with other elements, e.g., metals. Carbon nanotubes are generally about 0.5 to 2 nm in diameter where the ratio of the length dimension to the narrow dimension (diameter), i.e., the aspect ratio, is at least 5. Carbon nanotubes may be either multi-walled nanotubes or single-walled nanotubes. A multi-walled nanotube includes several concentric nanotubes, each having a different diameter. Thus, the smallest diameter tube is encapsulated by a larger diameter tube, which in turn, is encapsulated by another larger diameter nanotube. A single-walled nanotube, on the other hand, includes only one nanotube.

The term “subsequence” refers to a sequence of two to about five amino acid residues that are identified in the population of substrate-binding peptides.

The phrase “subsequences that occur statistically more frequently than by random chance” refers to subsequences that occur in the population of substrate-binding peptides with a frequency that is higher than that expected on the basis of random chance, as determined using statistical methods.

The phrase “statistically significant population of subsequences” refers to a population of subsequences that occurs statistically more frequently than by random chance.

The term “a hair-binding composition” refers to a composition for the treatment of hair comprising a hair-binding peptide coupled to a benefit agent. Compositions for the treatment of hair include, but not limited to, shampoos, conditioners, lotions, aerosols, gels, mousses, styling aids, hair straightening aids, hair strengthening aids, volumizing compositions and hair colorants.

The terms “coupling” and “coupled” as used herein refer to any chemical association and includes both covalent and non-covalent interactions.

The term “nanoparticles” is herein defined as particles with an average particle diameter of between 1 and 100 nm. Preferably, the average particle diameter of the particles is between about 1 and 40 nm. As used herein, “particle size” and “particle diameter” have the same meaning. Nanoparticles include, but are not limited to, metallic, semiconductor, polymer, or silica particles.

The phrase “method for modifying hair” refers to a method for treating hair, including, but not limited to, conditioning and coloring.

The term “stringency” as it is applied to the selection of substrate-binding peptides of the present invention, refers to the concentration of the eluting agent (usually detergent) used to elute peptides from the substrate. Higher concentrations of the eluting agent provide more stringent conditions.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

The term “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The term “host cell” refers to cell which has been transformed or transfected, or is capable of transformation or transfection by an exogenous polynucleotide sequence.

The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

“PCR” or “polymerase chain reaction” is a technique used for the amplification of specific DNA segments (U.S. Pat. Nos. 4,683,195 and 4,800,159).

Standard recombinant DNA and molecular cloning techniques used herein are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

The method of the invention provides a means for determining the sequence of a peptide binding motif having affinity for a particular substrate. First, a population of binding peptides for the substrate of interest is identified by biopanning using a combinatorial method, such as phage display. Rather than using many rounds of biopanning to identify specific binding peptide sequences and then using standard pattern recognition techniques to identify binding motifs, as is conventionally done in the art, the method of the invention requires only a few rounds of biopanning. The sequences in the population of binding peptides, which are generated by biopanning, are analyzed by identifying subsequences of 2, 3, 4, and 5 amino acid residues that occur more frequently than expected by random chance. The identified subsequences are then matched head to tail to give peptide motifs with substrate binding properties. This procedure may be repeated many times to generate long peptide sequences. Phage display alone is unlikely to identify long peptide sequences in which all the residues participate in binding. Moreover, the method is able to generate binding sequences that are not present in the initial library of sequences. Additionally, once specific surface binging motifs have been identified they may be used and reused to generate new surface binding peptides. Heretofore no method has been able to identify commonality in combinatorially generated surface binding peptides.

Population of Binding Peptides

A population of suitable substrate-binding peptide sequences may be generated using methods that are well known in the art. The peptides of the present invention are generated randomly and then selected against a specific substrate based upon their binding affinity for the substrate of interest. The generation of random libraries of peptides is well known and may be accomplished by a variety of techniques including, bacterial display (Kemp, D. J.; Proc. Natl. Acad. Sci. USA 78(7):4520-4524 (1981), and Helfman et al., Proc. Natl. Acad. Sci. USA 80(1):31-35, (1983)), yeast display (Chien et al., Proc Natl Acad Sci USA 88(21):9578-82 (1991)), combinatorial solid phase peptide synthesis (U.S. Pat. No. 5,449,754, U.S. Pat. No. 5,480,971, U.S. Pat. No. 5,585,275, U.S. Pat. No. 5,639,603), and phage display technology (U.S. Pat. No. 5,223,409, U.S. Pat. No. 5,403,484, U.S. Pat. No. 5,571,698, U.S. Pat. No. 5,837,500). Techniques to generate such biological peptide libraries are well known in the art. Exemplary methods are described in Dani, M., J. of Receptor & Signal Transduction Res., 21 (4):447-468 (2001), Sidhu et al., Methods in Enzymology 328:333-363 (2000), and Phage Display of Peptides and Proteins, A Laboratory Manual, Brian K. Kay, Jill Winter, and John McCafferty, eds.; Academic Press, NY, 1996. Additionally, phage display libraries may be purchased from New England BioLabs (Beverly, Mass.).

A preferred method to randomly generate peptides is by phage display. Phage display is an in vitro selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. In its simplest form, biopanning is carried out by incubating the pool of phage-displayed variants with a target of interest that has been immobilized on a plate or bead, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. In the method of the invention, only one or two rounds of biopanning are generally required to obtain the population of binding peptides.

Specifically, after a suitable library of peptides has been generated, they are then contacted with an appropriate amount of the test substrate. Exemplary test substrates include, but not limited to, body surfaces, such as hair, skin, nails, teeth, surfaces of the oral cavity, and corneal tissue; pigments, print media, such as printing paper, sheets, films, nonwovens and textile fabrics, such as polyester, nylon, Lycra®, silk, cotton, cotton blends, rayon, flax, linen, wool, spandex, acetate, acrylic, modacrylic, aramid and polyolefin; carbon nanotubes, semiconductors, and various polymers such as poly(methyl methacrylate) and poly(vinylidene chloride). These substrates are available commercially from various sources. For example, human hair samples are available commercially from International Hair Importers and Products (Bellerose, N.Y.), in different colors, such as brown, black, red, and blond, and in various types, such as African-American, Caucasian, and Asian. Additionally, the hair samples may be treated for example using hydrogen peroxide to obtain bleached hair. Pig skin, available from butcher shops and supermarkets, Vitro-Skin®, available from IMS Inc. (Milford, Conn.), and EpiDerm™, available from MatTek Corp. (Ashland, Mass.), are good substitutes for human skin. Human fingernails and toenails may be obtained from volunteers. The print media and polymers are also readily available from a number of commercial sources.

The library of peptides is dissolved in a suitable solution for contacting the substrate. A preferred solution is a buffered aqueous saline solution containing a surfactant. A suitable solution is Tris-buffered saline (TBS) with 0.5% Tween® 20. For contacting with the library of peptides, the substrate may be suspended in the solution or immobilized on a bead or plate. The solution may additionally be agitated by any means in order to increase the mass transfer rate of the peptides to the substrate, thereby shortening the time required to attain maximum binding.

Upon contact, a number of the randomly generated peptides will bind to the test substrate to form a peptide-substrate complex. Unbound peptide may be removed by washing. After all unbound material is removed, peptides having varying degrees of binding affinities for the test substrate may be fractionated by selected washings in elution buffers having varying stringencies. Increasing the stringency of the buffer used increases the required strength of the bond between the peptide and substrate in the peptide-substrate complex.

A number of substances may be used to vary the stringency of the buffer solution in peptide selection including, but not limited to, acids (pH 1.5-3.0); bases (pH 10-12.5); salts, such as MgCl₂ (3-5 M) and LiCl (5-10 M); water; ethylene glycol (25-50%); dioxane (5-20%); thiocyanate (1-5 M); guanidine (2-5 M); urea (2-8 M); and various concentrations of different surfactants such as SDS (sodium dodecyl sulfate), DOC (sodium deoxycholate), Nonidet P-40, Triton X-100, Tween® 20, wherein Tween® 20 is preferred. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is preferred.

It will be appreciated that peptides having increasing binding affinities for the test substrate may be eluted by repeating the selection process using buffers with increasing stringencies. The eluted peptides can be identified and sequenced by any means known in the art.

In one embodiment, the following phage display method may be used to generate a population of binding peptides. A library of combinatorially generated phage-peptides is contacted with the substrate of interest to form phage-peptide-substrate complexes. The phage-peptide-substrate complexes are separated from uncomplexed peptides and unbound substrate. Then, the bound phage-peptides are eluted from the complex, preferably by acid treatment. The eluted peptides are identified and sequenced.

To identify peptide sequences that bind to one substrate but not to another, for example peptides that bind to hair, but not to skin or peptides that bind to skin, but not to hair, a subtractive panning step may be added. Specifically, the library of combinatorial generated phage-peptides is first contacted with the non-target to remove phage-peptides that bind to it. Then, the non-binding phage-peptides are contacted with the desired substrate and the above process is followed. Alternatively, the library of combinatorial generated phage-peptides may be contacted with the non-target and the desired substrate simultaneously. Then, the phage-peptide-substrate complexes are separated from the phage-peptide-non-target complexes and the method described above is followed for the desired phage-peptide-substrate complexes.

Additionally, elution-resistant phage-peptides that remain bound to the substrate after contacting with a high stringency elution buffer may be identified and sequenced. For example, the remaining elution-resistant phage-peptide-substrate complexes may be used to directly infect a bacterial host cell, such as E. coli ER2738, as described by Huang et al. al. (copending and commonly owned U.S. patent application Ser. No. 10/935642 and U.S. Patent Application Publication No. 2005/0050656). The infected host cells are grown in a suitable growth medium, such as LB (Luria-Bertani) medium, and this culture is spread onto agar, containing a suitable growth medium, such as LB medium with IPTG (isopropyl β-D-thiogalactopyranoside) and S-GaI™. After growth, the plaques are picked for DNA isolation and sequencing to identify the peptide sequences with a high binding affinity for the substrate. Alternatively, the remaining bound phage-peptides may be amplified using a nucleic acid amplification technique, such as the polymerase chain reaction (PCR). In that approach, PCR is carried out on the remaining bound phage-peptides using the appropriate primers, as described by Janssen et al. in U.S. Patent Application Publication No. 2003/0152976, which is incorporated herein by reference.

The population of substrate-binding peptides consists of at least about 50 unique peptides, preferably at least about 75 unique peptides, more preferably, at least about 100 unique peptides.

Determination of the Frequency of Occurrence of Amino Acids in the Original Library

The frequency of occurrence of amino acids in the original library may be determined in any number of ways. For example, at least 50, preferably at least 100 random clones from the display library may be sequenced. The frequency of occurrence of each amino acid may be determined by dividing the number of times that particular amino acid is found in the sequences by the total number of amino acids sequenced. It is preferred to also examine the sequences of the random clones to determine if there is any non-random distribution of the amino acids in the random library clones. Such an examination may include determining if any amino acid occurs in a position in the sequences more or less frequently than would be expected from random chance, determining if any groups of amino acids, for example, hydrophobic, occur in a position in the sequences more or less frequently than would be expected from random chance, determining if runs of groups of amino acids, for example, hydrophobic, occur more or less frequently than would be expected from random chance, and determining, by methods described herein, if short subsequences of amino acids occur more frequently than would be expected from random chance. Alternatively, the frequency of occurrence of each amino acid may be obtained from the manufacturer of the display library or from published data.

Identification and Counting of Subsequences

The unique two to about five amino acid residue subsequences are identified in the population of substrate-binding peptides and the number of occurrences of each of the unique subsequences is determined and recorded. The identification and counting of the subsequences may be done in a number of ways. For example, the subsequences may be identified by visual inspection and counted manually. Alternatively, a computer program may be written in any suitable computer language to identify and count the number of occurrences of the unique subsequences. Additionally, a spreadsheet program, such as Excel® may be setup with macros to identify the unique subsequences and count the number of occurrences of the subsequences. An example of such an Excel® macro code is provided in Example 2, below.

Estimating the Probability of the Number of Occurrences of Each Subsequence

The probability of obtaining the number of subsequences that are observed is determined by first estimating the probability that a given sequence has the right amino acids to contain the subsequence. If an amino acid is not required in the subsequence, the fractional probability for that amino acid is assigned a value of 1. If one or more instances of an amino acid are required in the subsequence, the fractional probability (fp) for getting at least that many instances of that amino acid in a random sequence may be estimated by the binomial distribution, specifically:

$$fp = {\sum }_{x = 0}^{m} C ( n , x ) {{p}^{x} ( 1 - p )}^{n - x} \qquad ( 1 )$$

where n=the length of the sequence, m=the sequence length minus the number of occurrences of the amino acid required for the subsequence, x is the index having values from 0 to m, p=1 minus the fractional probability for the occurrence of that particular amino acid in the original library as determined as described above, and

$$C ( n , x ) = \frac{n !}{x ! ( n - x ) !} \qquad ( 2 )$$

The probability that a sequence contains at least the right number of amino acids (Ps) to make the subsequence is the product of the fractional probabilities for each amino acid (fp), as calculated using equation 1.

Next, the probability that the amino acids are arranged in the desired order, given that the sequence has the right amino acids, is estimated. This probability may be estimated by calculating the fraction of possible arrangements of the sequence that contain the subsequence. The amino acids in a peptide sequence of length n can be arranged in n! ways. Since only the unique sequences are of interest, this accounting may be corrected for multiple instances of amino acids in the subsequence as follows:

$${N}_{US} = \frac{n !}{{\prod }^{} j !} \qquad ( 3 )$$

where N_{US} is the number of unique sequences, n is the length of the sequence, j is the number of occurrences of each amino acid in the subsequence, and the π operator indexes through the 20 natural amino acids. The number of arrangements containing the subsequence is (n-I+1)! where n is the length of the sequence and I is the length of the subsequence. The probability that the amino acids are arranged in the correct order, given that the sequence contains the right amino acids, is

$${p}_{order} = \frac{( n - l + 1 )}{{N}_{US}} \qquad ( 4 )$$

Optionally, N_{US} may be further corrected to account for the sequence-containing amino acids in higher abundance that are required to form the subsequence. Another option is to further correct N_{US} to account for more than one instance of an amino acid in the sequence but outside the subsequence.

The next step is estimating the probability of the number of occurrences of each subsequence given the probability it will occur and the number of unique sequences that were identified and the length of those sequences. The probability of obtaining a specific subsequence (pₛₛ) in a random sequence is given by
pₛₛ=Ps×p_{order} (5)
The probability of obtaining at least m occurrences of a subsequence in n random clones (p_{occ}) where the probability of getting the subsequence in one random sequence is pₛₛ can be described by the binomial distribution as

$${p}_{occ} = {\sum }_{x = 0}^{m} C ( n , x ) {{p}^{x} ( 1 - p )}^{n - x} \qquad ( 6 )$$

where n=the number of random sequences, m=the number of occurrences of the subsequence in the n random sequences, x is the index having values from 0 to m, p=1−pₛₛ. and

$$C ( n , x ) = \frac{n !}{x ! ( n - x ) !} \qquad ( 7 )$$

To assess the likelihood that a subsequence is occurring more frequently than would be expected by random chance, the probability for each subsequence needs to be calculated if it occurs in the dataset more than once, or compared to a baseline if it occurs only once in the dataset. The baseline is a subsequence whose length is the same as the subsequence being evaluated. The amino acids in the baseline subsequence are preferably chosen from those whose frequency of occurrence is closest to that of the average rate of occurrence of 0.05. The number of occurrences of each subsequence is noted. If the subsequence occurs more than once, the probability of such an occurrence is calculated using equation 6. That probability should be less than about 0.2, preferably less than about 0.10, more preferably less than about 0.075, and most preferably less than about 0.05. If the subsequence occurs only once in the dataset, the probability for each such subsequence is compared to the baseline. Only subsequences whose probability is significantly less than that of the baseline sequence is carried forward in the analysis. The ratio of the baseline probability to the subsequence probability should be at least about 3, preferably at least about 5, more preferably at least about 10, and most preferably at least about 20. This means that the statistical probability of occurrence of the subsequence is at least about 3, preferably at least about 5, more preferably at least about 10, and most preferably at least about 20 times more frequent than by random chance.

Assembly of Subsequences

The remaining subsequences are tabulated, for example, in a list. Then, the first two amino acids of each subsequence and the last two amino acids of each subsequence are tabulated. While it is not necessary, it is helpful to classify the subsequences into 4 categories, Orphans, Sinks, Linkers, and Sources. Orphans are subsequences whose last two amino acids do not match any other subsequence's first two amino acids and whose first two amino acids do not match with any other subsequence's last two amino acids. Orphan subsequences are omitted from further consideration. Sinks are subsequences whose last two amino acids do not match any other subsequence's first two amino acids, but whose first two amino acids match with one or more other subsequence's last two amino acids. Sources are subsequences whose last two amino acids match with one or more other subsequence's first two amino acids, but whose first two amino acids do not match with any other subsequence's last two amino acids. Linkers are subsequences whose last two amino acids match with one or more other subsequence's first two amino acids and whose first two amino acids match with one or more other subsequence's last two amino acids.

Next, a non-Sink subsequence is selected as a starting point. It is preferred to start with a Source subsequence. The subsequences that have their first two amino acids match the last two amino acids of the starting subsequence are noted. A candidate sequence is formed by concatenating the amino acids of the matching subsequence starting with the third amino acid to the starting subsequence. If there is more than one other subsequence whose first two amino acids match the last amino acids of the starting subsequence, one is selected at random to use to begin.

The candidate sequence is used in a manner similar to the starting sequence. Specifically, other subsequences that have their first two amino acids match the last two amino acids of the candidate sequence are noted. The candidate sequence is extended by concatenating the amino acids of the matching subsequence, starting with the third amino acid, to the candidate sequence. If there is more than one other subsequence whose first two amino acids match the last amino acids of the candidate subsequence, one is selected at random for use. This method is continued to extend the candidate sequence until the desired sequence length is obtained or the matching process leads to a Sink subsequence. The generated peptide motifs have a length of at least 3 amino acids, preferably, 3 to about 50 amino acids.

Additional sequences may be generated by starting with a different subsequence or by starting with the same subsequence and, where choices between matches were made, choosing different matches than were chosen previously. This process is continued until the number of sequences desired is obtained or all possible combination matches have been used.

It is possible to concatenate two or more sequences generated in the manner described above with or without additional amino acids to separate the sequences.

Production of Candidate Binding Peptides

The candidate binding peptides, generated as described above, may be prepared using standard peptide synthesis methods, which are well known in the art (see for example Stewart et al., Solid Phase Peptide Synthesis, Pierce Biotechnology, Inc., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the candidate binding peptides may be prepared using recombinant DNA and molecular cloning techniques. Genes encoding the candidate binding peptides may be produced in heterologous host cells, particularly in the cells of microbial hosts. Preferred heterologous host cells for expression of candidate binding peptides of the present invention are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, fungal or yeast species such as Aspergillus, Trichoderma, Saccharomyces, Pichia, Candida, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

A variety of expression systems can be used to produce the peptides of the present invention. Such vectors include, but are not limited to, chromosomal, episomal and virus-derived vectors, e.g., vectors derived from bacterial plasmids, from bacteriophage, from transposons, from insertion elements, from yeast episoms, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these could be used to construct chimeric genes for production of the any of the binding peptides of the present invention. These chimeric genes could then be introduced into appropriate microorganisms via transformation to provide high level expression of the peptides.

Vectors or cassettes useful for the transformation of suitable host cells are well known in the art. Typically the vector or cassette contains sequences directing transcription and translation of the relevant gene, one or more selectable markers, and sequences allowing autonomous replication or chromosomal integration. Suitable vectors comprise a region 5′ of the gene, which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Selectable marker genes provide a phenotypic trait for selection of the transformed host cells such as tetracycline or ampicillin resistance in E. coli.

Initiation control regions or promoters which are useful to drive expression of the chimeric gene in the desired host cell are numerous and familiar to those skilled in the art. Virtually any promoter capable of driving the gene is suitable for producing the binding peptides of the present invention including, but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

The vector containing the appropriate DNA sequence as described supra, as well as an appropriate promoter or control sequence, may be employed to transform an appropriate host to permit the host to express the peptide of the present invention. Cell-free translation systems can also be employed to produce such peptides using RNAs derived from the DNA constructs of the present invention. Optionally it may be desired to produce the instant gene product as a secretion product of the transformed host. Secretion of desired product into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the production host. Methods for choosing appropriate signal sequences are well known in the art (see for example EP 546049 and WO 9324631). The secretion signal DNA or facilitator may be located between the expression-controlling DNA and the instant gene or gene fragment, and in the same reading frame with the latter.

After the desired peptide sequences have been produced, they are optionally screened for substrate binding activity using methods known in the art, such as an enzyme-linked immunoassay (ELISA) method or a radiochemical method. The candidate peptide sequences that exhibit strong, specific binding to the desired substrate may then be used for the intended purpose, for example, for the preparation of hair binding compositions, as described by Huang et al. (copending and commonly owned U.S. patent application Ser. No. 10/935642 and U.S. Patent Application Publication No. 2005/0050656) or peptide-based diblock and triblock dispersants and diblock polymers, as described by Obrien et al. (copending and commonly owned U.S. patent application Ser. No. 10/935254 and U.S. Patent Application Publication No. 2005/0054752), all of which are incorporated herein by reference.

Hair Binding Compositions

The method of the invention was used to generate hair-binding peptide motifs for use in hair binding compositions, including, but not limited to, shampoos, conditioners, lotions, aerosols, gels, mousses, styling aids, hair straightening aids, hair strengthening aids, volumizing compositions and hair colorants. The hair-binding peptide motifs generated using the method of the invention have the sequences given by SEQ ID NOs:81-123. These hair-binding peptides may be used to prepare peptide-based hair colorants and hair conditioners, as described by Huang et al., supra. As described therein, the peptide-based hair conditioners or hair colorants are formed by coupling a hair-binding peptide (HBP) to a hair conditioning agent (HCA) or a coloring agent (C), respectively. The hair-binding peptide binds strongly to the hair, thus keeping the conditioning agent or coloring agent attached to the hair for a long lasting effect.

In the peptide-based hair conditioners and hair colorants of the invention, any suitable hair conditioning agent or coloring agent may be used. Hair conditioning agents, as herein defined, are agents which improve the appearance, texture, and sheen of hair as well as increasing hair body or suppleness. Hair conditioning agents, include, but are not limited to, styling aids, hair straightening aids, hair strengthening aids, and volumizing agents, such as nanoparticles. Hair conditioning agents are well known in the art, see for example Green et al. (WO 0107009, in particular, page 44 line 11 to page 68 line 14), incorporated herein by reference, and are available commercially from various sources. Suitable examples of hair conditioning agents include, but are not limited to, cationic polymers, such as cationized guar gum, diallyl quaternary ammonium salt/acrylamide copolymers, quaternized polyvinylpyrrolidone and derivatives thereof, and various polyquaternium-compounds; cationic surfactants, such as stearalkonium chloride, centrimonium chloride, and Sapamin hydrochloride; fatty alcohols, such as behenyl alcohol; fatty amines, such as stearyl amine; waxes; esters; nonionic polymers, such as polyvinylpyrrolidone, polyvinyl alcohol, and polyethylene glycol; silicones; siloxanes, such as decamethylcyclopentasiloxane; polymer emulsions, such as amodimethicone; and nanoparticles, such as silica nanoparticles and polymer nanoparticles. The preferred hair conditioning agents of the present invention contain amine or hydroxyl functional groups to facilitate coupling to the hair-binding peptides, as described below. Examples of preferred conditioning agents are octylamine (CAS No.111-86-4), stearyl amine (CAS No.124-30-1), behenyl alcohol (CAS No. 661-19-8, Cognis Corp., Cincinnati, Ohio), vinyl group terminated siloxanes, vinyl group terminated silicone (CAS No. 68083-19-2), vinyl group terminated methyl vinyl siloxanes, vinyl group terminated methyl vinyl silicone (CAS No. 68951-99-5), hydroxyl terminated siloxanes, hydroxyl terminated silicone (CAS No. 80801-30-5), amino-modified silicone derivatives, [(aminoethyl)amino]propyl hydroxyl dimethyl siloxanes, [(aminoethyl)amino]propyl hydroxyl dimethyl silicones, and alpha-tridecyl-omega-hydroxy-poly(oxy-1,2-ethanediyl) (CAS No. 24938-91-8).

Coloring agents as herein defined are any dye, pigment, and the like that may be used to change the color of hair. Hair coloring agents are well known in the art (see for example Green et al. supra (in particular, page 42 line 1 to page 44 line 11), CFTA International Color Handbook, 2^{nd} ed., Micelle Press, England (1992) and Cosmetic Handbook, US Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany). Suitable hair coloring agents include, but are not limited to dyes, such as 4-hydroxypropylamino-3-nitrophenol, 4-amino-3-nitrophenol, 2-amino-6-chloro-4-nitrophenol, 2-nitro-paraphenylenediamine, N,N-hydroxyethyl-2-nitro-phenylenediamine, 4-nitro-indole, Henna, HC Blue 1, HC Blue 2, HC Yellow 4, HC Red 3, HC Red 5, Disperse Violet 4, Disperse Black 9, HC Blue 7, HC Blue 12, HC Yellow 2, HC Yellow 6, HC Yellow 8, HC Yellow 12, HC Brown 2, D&C Yellow 1, D&C Yellow 3, D&C Blue 1, Disperse Blue 3, Disperse violet 1, eosin derivatives such as D&C Red No. 21 and halogenated fluorescein derivatives such as D&C Red No. 27, D&C Red Orange No. 5 in combination with D&C Red No. 21 and D&C Orange No. 10; and pigments, such as D&C Red No. 36 and D&C Orange No. 17, the calcium lakes of D&C Red Nos. 7, 11, 31 and 34, the barium lake of D&C Red No. 12, the strontium lake of D&C Red No. 13, the aluminum lakes of FD&C Yellow No. 5, of FD&C Yellow No. 6, of D&C Red No. 27, of D&C Red No. 21, and of FD&C Blue No. 1, iron oxides, manganese violet, chromium oxide, titanium dioxide, titanium dioxide nanoparticles, zinc oxide, barium oxide, ultramarine blue, bismuth citrate, and carbon black particles. The preferred hair coloring agents of the present invention are D&C Yellow 1 and 3, HC Yellow 6 and 8, D&C Blue 1, HC Blue 1, HC Brown 2, HC Red 5, 2-nitro-paraphenylenediamine, N,N-hydroxyethyl-2-nitro-phenylenediamine, 4-nitro-indole, and carbon black.

Metallic and semiconductor nanoparticles may also be used as hair coloring agents due to their strong emission of light (Vic et al. U.S. Patent Application Publication No. 2004/0010864). The metallic and semiconductor nanoparticles may also serve as volumizing agents, as described above.

Additionally, the coloring agent may be a colored, polymeric microsphere. Exemplary polymeric microspheres include, but are not limited to, microspheres of polystyrene, polymethylmethacrylate, polyvinyltoluene, styrene/butadiene copolymer, and latex. For use in the invention, the microspheres have a diameter of about 10 nanometers to about 2 microns. The microspheres may be colored by coupling any suitable dye, such as those described above, to the microspheres. The dyes may be coupled to the surface of the microsphere or adsorbed within the porous structure of a porous microsphere. Suitable microspheres, including undyed and dyed microspheres that are functionalized to enable covalent attachment, are available from companies such as Bang Laboratories (Fishers, Ind.).

The peptide-based hair conditioners or hair colorants of the invention are prepared by coupling a specific hair-binding peptide to a hair conditioning agent or a coloring agent, either directly or via an optional spacer. The coupling interaction may be a covalent bond or a non-covalent interaction, such as hydrogen bonding, electrostatic interaction, hydrophobic interaction, or Van der Waals interaction. In the case of a non-covalent interaction, the peptide-based hair conditioner or colorant may be prepared by mixing the peptide with the conditioning agent or coloring agent and the optional spacer (if used) and allowing sufficient time for the interaction to occur. The unbound materials may be separated from the resulting peptide-based hair conditioner or hair colorant adduct using methods known in the art, for example, gel permeation chromatography.

The peptide-based hair conditioners or hair colorants of the invention may also be prepared by covalently attaching a specific hair-binding peptide to a hair conditioning agent or coloring agent, either directly or through a spacer. Any known peptide or protein conjugation chemistry may be used to form the peptide-based hair conditioners or hair colorants. Conjugation chemistries are well-known in the art (see for example, Hermanson, Bioconjugate Techniques, Academic Press, New York (1996)). Suitable coupling agents include, but are not limited to, carbodiimide coupling agents, diacid chlorides, diisocyanates and other difunctional coupling reagents that are reactive toward terminal amine and/or carboxylic acid terminal groups on the peptides and to amine, carboxylic acid, or alcohol groups on the hair conditioning agent or coloring agent. The preferred coupling agents are carbodiimide coupling agents, such as 1-ethyl-3-(3-dimethylaminopropyl)-carbodiimide (EDC) and N,N′-dicyclohexyl-carbodiimide (DCC), which may be used to activate carboxylic acid groups for coupling to alcohol, and amine groups. Additionally, it may be necessary to protect reactive amine or carboxylic acid groups on the peptide to produce the desired structure for the peptide-based hair conditioner or hair colorant. The use of protecting groups for amino acids, such as t-butyloxycarbonyl (t-Boc), are well known in the art (see for example Stewart et al., supra; Bodanszky, supra; and Pennington et al., supra). In some cases it may be necessary to introduce reactive groups, such as carboxylic acid, alcohol, amine, or aldehyde groups, on the hair conditioning agent or coloring agent for coupling to the hair-binding peptide. These modifications may be done using routine chemistry such as oxidation, reduction and the like, which is well known in the art.

It may also be desirable to couple the hair-binding peptide to the hair conditioning agent or coloring agent via a spacer. The spacer serves to separate the conditioning agent or coloring agent from the peptide to ensure that the agent does not interfere with the binding of the peptide to the hair. The spacer may be any of a variety of molecules, such as alkyl chains, phenyl compounds, ethylene glycol, amides, esters and the like. Preferred spacers are hydrophilic and have a chain length from 1 to about 100 atoms, more preferably, from 2 to about 30 atoms. Examples of preferred spacers include, but are not limited to, ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, and ethyl, propyl, hexyl, steryl, cetyl, and palmitoyl alkyl chains. The spacer may be covalently attached to the peptide and the hair conditioning agent or coloring agent using any of the coupling chemistries described above. In order to facilitate incorporation of the spacer, a bifunctional cross-linking agent that contains a spacer and reactive groups at both ends for coupling to the peptide and the conditioning agent or the coloring agent may be used. Suitable bifunctional cross-linking agents are well known in the art and include, but are not limited to, diamines, such a as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyantes, such as hexamethylenediisocyanate; bis oxiranes, such as 1,4 butanediyl diglycidyl ether; dicarboxylic acids, such as succinyldisalicylate; and the like. Heterobifunctional cross-linking agents, which contain a different reactive group at each end, may also be used. Examples of heterobifunctional cross-linking agents include, but are not limited to compounds having the following structure: 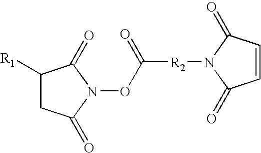
where: R₁ is H or a substituent group such as —SO₃Na, —NO₂, or —Br; and R₂ is a spacer such as —CH₂CH₂ (ethyl), —(CH₂)₃ (propyl), or —(CH₂)₃C₆H₅ (propyl phenyl). An example of such a heterobifunctional cross-linking agent is 3-maleimidopropionic acid N-hydroxysuccinimide ester. The N-hydroxysuccinimide ester group of these reagents reacts with amine or alcohol groups on the hair conditioning agent or coloring agent, while the maleimide group reacts with thiol groups present on the peptide. A thiol group may be incorporated into the peptide by adding a cysteine group to at least one end of the binding peptide sequence (i.e., the C-terminus or N-terminus). Several spacer amino acid residues, such as glycine, may be incorporated between the binding peptide sequence and the terminal cysteine to separate the reacting thiol group from the binding sequence.

Additionally, the spacer may be a peptide composed of any amino acid and mixtures thereof. The preferred peptide spacers are composed of the amino acids glycine, alanine, lysine, and serine, and mixtures thereof. In addition, the peptide spacer may contain a specific enzyme cleavage site, such as the protease Caspase 3 site, given by SEQ ID NO:125, which allows for the enzymatic removal of the conditioning agent from the hair. The peptide spacer may be from 1 to about 50 amino acids, preferably from 1 to about 20 amino acids. These peptide spacers may be linked to the binding peptide sequence by any method known in the art. For example, the entire binding peptide-peptide spacer diblock may be prepared using the standard peptide synthesis methods described supra. In addition, the binding peptide and peptide spacer blocks may be combined using carbodiimide coupling agents (see for example, Hermanson, Bioconjugate Techniques, Academic Press, New York (1996)), diacid chlorides, diisocyanates and other difunctional coupling reagents that are reactive to terminal amine and/or carboxylic acid terminal groups on the peptides. Alternatively, the entire binding peptide-peptide spacer diblock may be prepared using the recombinant DNA and molecular cloning techniques described supra. The spacer may also be a combination of a peptide spacer and an organic spacer molecule, which may be prepared using the methods described above.

It may also be desirable to have multiple hair-binding peptides coupled to the hair conditioning agent or coloring agent to enhance the interaction between the peptide-based hair conditioner or colorant and the hair. Either multiple copies of the same hair-binding peptide or a combination of different hair-binding peptides may be used.

The peptide-based hair conditioners may be used in compositions for hair care. It should also be recognized that the hair-binding peptides themselves can serve as conditioning agents for the treatment of hair. Hair care compositions are herein defined as compositions for the treatment of hair, including but not limited to shampoos, conditioners, lotions, aerosols, gels, mousses, and hair dyes comprising an effective amount of a peptide-based hair conditioner or a mixture of different peptide-based hair conditioners in a cosmetically acceptable medium. An effective amount of a peptide-based hair conditioner or hair-binding peptide for use in a hair care composition is herein defined as a proportion of from about 0.01% to about 10%, preferably about 0.01% to about 5% by weight relative to the total weight of the composition. Components of a cosmetically acceptable medium for hair care compositions are described by Philippe et al. in U.S. Pat. No. 6,280,747, and by Omura et al. in U.S. Pat. No. 6,139,851 and Cannell et al. in U.S. Pat. No. 6,013,250, all of which are incorporated herein by reference. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hair care compositions may contain one or more conventional cosmetic or dermatological additives or adjuvants including but not limited to, antioxidants, preserving agents, fillers, surfactants, UVA and/or UVB sunscreens, fragrances, thickeners, wetting agents and anionic, nonionic or amphoteric polymers, and dyes or pigments.

The peptide-based hair colorants may be used in hair coloring compositions for dyeing hair. Hair coloring compositions are herein defined as compositions for the coloring, dyeing, or bleaching of hair, comprising an effective amount of peptide-based hair colorant or a mixture of different peptide-based hair colorants in a cosmetically acceptable medium. An effective amount of a peptide-based hair colorant for use in a hair coloring composition is herein defined as a proportion of from about 0.001% to about 20% by weight relative to the total weight of the composition. Components of a cosmetically acceptable medium for hair coloring compositions are described by Dias et al., in U.S. Pat. No. 6,398,821 and by Deutz et al., in U.S. Pat. No. 6,129,770, both of which are incorporated herein by reference. For example, hair coloring compositions may contain sequestrants, stabilizers, thickeners, buffers, carriers, surfactants, solvents, antioxidants, polymers, and conditioners. The conditioners may include the peptide-based hair conditioners and hair-binding peptides of the present invention in a proportion from about 0.01% to about 10%, preferably about 0.01% to about 5% by weight relative to the total weight of the hair coloring composition.

The peptide-based hair colorants of the present invention may also be used as coloring agents in cosmetic compositions that are applied to the eyelashes or eyebrows including, but not limited to mascaras, and eyebrow pencils. These may be anhydrous make-up products comprising a cosmetically acceptable medium which contains a fatty substance in a proportion generally of from about 10 to about 90% by weight relative to the total weight of the composition, where the fatty phase containing at least one liquid, solid or semi-solid fatty substance, as described above. The fatty substance includes, but is not limited to, oils, waxes, gums, and so-called pasty fatty substances. Alternatively, these compositions may be in the form of a stable dispersion such as a water-in-oil or oil-in-water emulsion, as described above. In these compositions, the proportion of the peptide-based hair colorant is generally from about 0.001% to about 20% by weight relative to the total weight of the composition.

Methods for Modifying Hair

In another embodiment, methods are provided for modifying hair, with the hair binding compositions of the invention. Specifically, the present invention also comprises a method for conditioning or coloring hair by applying one of the compositions described above comprising an effective amount of a peptide-based hair conditioner or hair colorant to the hair. The compositions may be applied to the hair by various means, including, but not limited to spraying, brushing, and applying by hand. The hair binding composition is left in contact with the hair for a period of time sufficient to condition or color the hair, typically for at least about 5 seconds to about 50 minutes, and more preferably from about 5 seconds to about 60 seconds.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmole” means micromole(s), “g” means gram(s), “μg” means microgram(s), “mg” means milligram(s), “pfu” means plague forming unit, “BSA” means bovine serum albumin, “ELISA” means enzyme linked immunosorbent assay, “A” means absorbance, “A₄₅₀” means the absorbance measured at a wavelength of 450 nm, “TBS” means Tris-buffered saline, “TBST-X” means Tris-buffered saline containing Tween® 20 where “X” is the weight percent of Tween® 20, “IPTG” means isopropyl β-D-thiogalactoside, and “S-GaI™” means 3,4-cyclohexenoesculetin-β-D-galactopyranoside,

General Methods:

Standard recombinant DNA and molecular cloning techniques used in the Examples are well known in the art and are described by Sambrook, J., Fritsch, E. F. and Maniatis, T., Molecular Cloning: A Laboratory Manual, Cold Spring Harbor Laboratory Press, Cold Spring Harbor, N.Y., 1989, by T. J. Silhavy, M. L. Bennan, and L. W. Enquist, Experiments with Gene Fusions, Cold Spring Harbor Laboratory, Cold Spring Harbor, N.Y., 1984, and by Ausubel, F. M. et al., Current Protocols in Molecular Biology, Greene Publishing Assoc. and Wiley-Interscience, N.Y., 1987. All reagents and materials used in the following examples were obtained from Aldrich Chemicals (Milwaukee, Wis.), BD Diagnostic Systems (Sparks, Md.), Life Technologies (Rockville, Md.), or Sigma Chemical Company (St. Louis, Mo.), unless otherwise specified.

### Example 1

### Generation of a Population of Hair-Binding Peptides

The purpose of this Example was to generate a population of hair-binding phage peptides that bind to bleached hair using standard phage display biopanning.

Phase Display Peptide Libraries:

The phage library used in this Example, Ph.D.-12™ Phage Display Peptide Library Kit, was purchased from New England BioLabs (Beverly, Mass.). This kit is based on a combinatorial library of random peptide 12-mers fused to a minor coat protein (pIII) of M13 phage. The displayed peptide is expressed at the N-terminus of pIII, such that after the signal peptide is cleaved, the first residue of the coat protein is the first residue of the displayed peptide. The Ph.D.-12 library consist of 2.7×10⁹ sequences. A volume of 10 μL contains about 55 copies of each peptide sequence. Each initial round of experiments was carried out using the original library provided by the manufacture in order to avoid introducing any bias into the results.

Preparation of Hair Samples:

The hair samples used were 6-inch (15.2 cm) medium brown human hairs obtained from International Hair Importers and Products (Bellerose, N.Y.). The hairs were placed in 90% isopropanol for 30 min at room temperature and then washed 5 times for 10 min each with deionized water. The hairs were air-dried overnight at room temperature. To prepare the bleached hair samples, the air-dried medium brown human hairs were placed in 6% H₂O₂, which was adjusted to pH 10.2 with ammonium hydroxide, for 10 min at room temperature and then washed 5 times for 10 min each with deionized water. The hairs were air-dried overnight at room temperature.

The bleached hair samples were cut into 0.5 to 1 cm lengths and about 5 to 10 mg of the hairs was placed into wells of a custom 24-well biopanning apparatus that had a pig skin bottom. An equal number of the pig skin bottom wells were left empty. The pig skin bottom apparatus was used as a subtractive procedure to remove phage-peptides that have an affinity for skin. This apparatus was created by modifying a dot blot apparatus (obtained from Schleicher & Schuell, Keene, N.H.) to fit the biopanning process. Specifically, the top 96-well block of the dot blot apparatus was replaced by a 24-well block. A 4×6 inch (10.2×15.2 cm) treated pig skin was placed under the 24-well block and panning wells with a pig skin bottom were formed by tightening the apparatus. The pig skin was purchased from a local supermarket and stored at −80 ° C. Before use, the skin was placed in deionized water to thaw, and then blotted dry using a paper towel. The surface of the skin was wiped with 90% isopropanol, and then rinsed with deionized water. The 24-well apparatus was filled with blocking buffer consisting of 1 mg/mL BSA in TBST containing 0.5% Tween® 20 (TBST-0.5%) and incubated for 1 h at 4° C. The wells and hairs were washed 5 times with TBST-0.5%. One milliliter of TBST-0.5% containing 1 mg/mL BSA was added to each well. Then, 10 μL of the original phage library (2×10¹¹ pfu) was added to the pig skin bottom wells that did not contain a hair sample and the phage library was incubated for 15 min at room temperature. The unbound phages were then transferred to pig skin bottom wells containing the hair samples and were incubated for 15 min at room temperature. The hair samples and the wells were washed 10 times with TBST-0.5%. The hairs were then transferred to clean, plastic bottom wells of a 24-well plate and 1 mL of a non-specific elution buffer consisting of 1 mg/mL BSA in 0.2 M glycine-HCl, pH 2.2, was added to each well and incubated for 10 min to elute the bound phages. The hairs that were treated with the acidic elution buffer were washed three more times with the elution buffer and then washed three times with TBST-0.5%. These hairs, which had acid resistant phage peptides still attached, were used to directly infect 500 μL of mid-log phase bacterial host cells, E. coli ER2738 (New England BioLabs). The cells were then grown in LB (Luria-Bertani) medium for 20 min and then mixed with 3 mL of agarose top (LB medium with 5 mM MgCl₂, and 0.7% agarose) at 45° C. This mixture was spread onto a LB medium/IPTG/S-GaI™ plate (LB medium with 15 g/L agar, 0.05 g/L IPTG, and 0.04 g/L S-GaI™) and incubated overnight at 37° C. The black plaques were counted to calculate the phage titer. The single black plaques were randomly picked for DNA isolation and sequencing analysis.

The single plaque lysates were prepared following the manufacture's instructions (New England Labs) and the single stranded phage genomic DNA was purified using the QIAprep Spin M13 Kit (Qiagen, Valencia, Calif.) and sequenced at the DuPont Sequencing Facility using −96 gIII sequencing primer (5′-CCCTCATAGTTAGCGTAACG-3′), given as SEQ ID NO:126. The displayed peptide is located immediately after the signal peptide of gene III.

The amino acid sequences of the acid resistant, bleached hair-binding phage peptides are given in Table 1.

TABLE 1

Population of Bleached Hair-Binding
Peptide Sequences

| | |
|---|---|
| Amino Acid | |
| Sequence | SEQ ID NO: |

| | |
|---|---|
| AETVESDLAKSH | 1 |
| AKPISQHLQRGS | 2 |
| ALKQDNTILLRE | 3 |
| ANLQRMTPSSLL | 4 |
| ANVQSHVDFQTR | 5 |
| ASQTQNVRHSWP | 6 |
| ASSDHHIPHSST | 7 |
| AYFPYPLSTYRF | 8 |
| DDFAKPYFSDTR | 9 |
| DHHKSNTLGQAS | 10 |
| DHRICMKTSPPL | 11 |
| DPRSTHLFVQSG | 12 |
| DSTYKVSNRSLQ | 13 |
| DSYDSNMFPPYI | 14 |
| EQISGSLVAAPW | 15 |
| ESQSRQESLQIA | 16 |
| FASGEHHTSPMD | 17 |
| FSFENFLSDRSH | 18 |
| GKAFVNQVRSSA | 19 |
| GRRLLLRLTPGG | 20 |
| GYSPIKRPPLDC | 21 |
| HHSSRYSDVLAV | 22 |
| HISPGWSPHRSD | 23 |
| HNQSRYYTGKLH | 24 |
| HQLSVRDWPLST | 25 |
| HRQTSLPSPIAR | 26 |
| HTPKNLSAPLTH | 27 |
| IHKPNLRATPFS | 28 |
| ITNSPSMHWSTF | 29 |
| IVHQLQTRPIKP | 30 |
| KIVNTYNRLQNL | 31 |
| KLKHNHIPDPYL | 32 |
| KNVDQSLRSFIV | 33 |
| KQVEHVTTRTLT | 34 |
| LDTSFPPVPFHA | 35 |
| LGHTTGVNIYSP | 36 |
| LMPPPWLGIASW | 37 |
| LPKTTNPLLRAH | 38 |
| LPLFPRELSVFT | 39 |
| LPVRNMLQERWP | 40 |
| NEVPARNAPWLV | 41 |
| NITTPTFKSIPM | 42 |
| NPPHPLALQQLR | 43 |
| QLIPHAHVRPPA | 44 |
| QSDYSGRLLGLG | 45 |
| SDLPGLANSPAH | 46 |
| SHISTSGPSPFG | 47 |
| SKWLSHYSDMLI | 48 |
| SLAPPVFMKFLK | 49 |
| SLNWVTIPGPKI | 50 |
| SMAHDPMAVRVY | 51 |
| SNAHPLTRVLLA | 52 |
| SNIQPQGTHWKT | 53 |
| SNTTPSPTPHKP | 54 |
| SPNPVTQNLIHT | 55 |
| SSYEFDMSAVEP | 56 |
| TAKWISGIDAPP | 57 |
| THHKTPLHHHRT | 58 |
| THPRSNTTASSG | 59 |
| TLTSVTVRQPLF | 60 |
| TLVIQPSLRLAS | 61 |
| TPHSEKTVVLNS | 62 |
| TPYWQTSTGTPE | 63 |
| TQDSAQKSPSPL | 64 |
| TQVPSPTHPAAF | 65 |
| TYTKAATETFEL | 66 |
| VHKPNIPPARNT | 67 |
| VKPPLDPIHASW | 68 |
| VPPSQPKQPNAL | 69 |
| VSVKMPYNYVAY | 70 |
| VVHTHATLGQAT | 71 |
| WDTCCYNNHPMP | 72 |
| WHAQFTPQPLSQ | 73 |
| WSDSGLNHPRMR | 74 |
| YNDFVNGHNPRT | 75 |
| YPVPYQTHHMVQ | 76 |
| YSQIPFAGPYTV | 77 |
| YTHDHRLHPRLL | 78 |
| YTTVNDAETPGH | 79 |
| YTVHTVDPHSHQ | 80 |

### Example 2

### Analysis of the Population of Bleached Hair Binding Peptides for Frequently Occurring Subsequences

The purpose of this Example was to identify and count the unique 3, 4, and 5 amino acid residue subsequences in the population of bleached hair-binding peptide sequences, given in Table 1, and to estimate the probability of the number of occurrences of each subsequence.

The unique subsequences were identified and counted using a macro in the spreadsheet program Excel®. The macro code used to accomplish this is given below.

```
Sub aa_sub_sequences( )
‘
‘ Select sheet for results and clear any previous results
‘
Sheets(“aa sub sequences”).Select
clear_sub
‘ nseq is the number of sequences being analyzed
‘
For iseq = 1 To nseq
  For sublength = 2 To 5
‘
‘ sublength is the length of subsequence being compiled
‘ seq$ is an array containing the sequences being analyzed
‘
    seqlength = Len(seq$(iseq))
    For i = 1 To seqlength − sublength + 1
      s$ = Mid$(seq$(iseq), i, sublength)
      ‘ look in the right table
      ‘ get number of table entries
      nentries = ActiveCell.Offset(0, (sublength − 1) * 4 − 3).Value
      If nentries = 0 Then
      Call add_entry(s$, sublength, nentries)
      Else
        imatch = False
        For n = 1 To nentries
          If s$ = ActiveCell.Offset(n + 2, (sublength − 1) *
          4 − 3).Value
Then
            imatch = True
            Exit For
          End If
        Next n
        If imatch Then
        ‘incrment subsequence counter
        ActiveCell.Offset(n + 2, (sublength − 1) * 4 − 2).Formula = _{—}
          ActiveCell.Offset(n + 2, (sublength − 1) * 4 − 2).Value + 1
        Else
        Call add_entry(s$, sublength, nentries)
        End If
      End If
    Next i
  Next sublength
Next iseq
sort_sub
End Sub
Sub add_entry(s$, sublength, nentries)
ActiveCell.Offset(nentries + 3, (sublength − 1) * 4 − 3).Formula = s$
ActiveCell.Offset(nentries + 3, (sublength − 1) * 4 − 2).Formula = 1
ActiveCell.Offset(0, (sublength − 1) * 4 − 3).Formula = nentries + 1
End Sub
Sub clear_sub( )
‘
‘ clears previous results from aa sub sequences sheet
‘
Range(“a1”).Select
Max = 0
For i = 2 To 14 Step 4
  If ActiveCell.Offset(0, i − 1).Value > Max Then Max =
ActiveCell.Offset(0, i − 1).Value
  ActiveCell.Offset(0, i − 1).Formula = 0
Next i
ActiveCell.Range(“a4:Q” & Trim$(Str(Max + 3))).Clear
End Sub
Sub sort_sub( )
‘
‘ sorts results in descending order
‘
For k = 2 To 14 Step 4
  Range(Cells(4, k), Cells(4, k + 1).End(xlDown)).Select
  Selection.Sort Key1:=Range(Cells(4, k + 1), Cells(4, k + 1)),
Order1:=xlDescending, Header:=xlNo, _{—}
    OrderCustom:=1, MatchCase:=False, Orientation:=xlTopToBottom
Next k
End Sub
```

The probability of obtaining the number of subsequences that were observed was calculated using equations 1-7, as described above. By way of example, the subsequence HKP was found three times in the population of 80 sequences (Table 1). The fraction probability that a sequence contained H, K and P was estimated using equation 1. The probability that a sequence contained at least 1 histidine was 0.5419. The probability that it contained at least one lysine or one proline was 0.2887 and 0.7901, respectively. The probability that a 12-mer sequence contained H, K and P was calculated from the product of these probabilities to be about 0.1237. The residues in a 12-mer peptide, having at least one instance of each H, K and P, can be rearranged into approximately 479 million sequences. Approximately 3.6 million of those sequences would contain the subsequence HKP. Thus, the probability that any 12-mer sequence from the library contains HKP was calculated to be:
0.1237×3.6×10⁶/479×10⁶=9.369×10⁻⁴

Knowing that probability and given that 3 instances of HKP were found in the population of 80 sequences, equation 6 was used to obtain the probability of such an occurrence, which was calculated to be 6.4×10⁻⁵.

The frequency of occurrence of amino acids in the original library was determined from data provided by the vendor (New England Biolabs) for the phage library. The values obtained from the vendor were verified by sequencing 80 random clones from the phage library. The frequency of occurrence of amino acids in the original library used in the calculations, given in Table 2, was the average of the data obtained from the vendor and the data obtained from sequencing. Given the frequency of occurrence of amino acids in the phage library, the reference sequence was taken as AHQRN.

TABLE 2
Frequency of Occurrence of Amino Acids in the Original Library

| Amino Acid | Average Occurrence in Library % |
|---|---|
| A | 6.0 |
| C | 0.5 |
| D | 2.8 |
| E | 3.1 |
| F | 3.3 |
| G | 2.6 |
| H | 6.3 |
| I | 3.4 |
| K | 2.8 |
| L | 9.3 |
| M | 2.6 |
| N | 4.6 |
| P | 12.2 |
| Q | 5.1 |
| R | 4.7 |
| S | 10.0 |
| T | 11.1 |
| V | 3.9 |
| W | 2.2 |
| Y | 3.6 |

The subsequences and the number of occurrences of each subsequence (N) were tabulated. Table 3 shows the number of unique subsequences found as a function of subsequence length. The reference sequences used to calculate the relative probabilities and the probability of those reference sequences are also shown in Table 3. The tabulation of subsequences having three amino acids and the number of occurrences for each subsequence are given in Table 4, which is sorted by relative probability in descending order. Only those subsequences that occurred more than once and had a probability of less than 0.075, or occurred once and had a relative probability greater than 10 are shown in the table.

TABLE 3
Number of Unique Subsequences Found as a
Function of Subsequence Length

| | | | Probability of |
|---|---|---|---|
| | | | Reference |
| Subsequence | Number of Unique | Reference | Subsequence |
| Length | Subsequences | Subsequence | (one occurrence) |
| 3 | 710 | AHQ | 0.07719 |
| 4 | 712 | AHQR (SEQ ID | 0.003517 |
| | | NO: 127) | |
| 5 | 639 | AHQRN (SEQ | 0.000169 |
| | | ID NO: 128) | |

TABLE 4
Unique Subsequences of Three Amino Acids Found and Their Probability
of Occurrence

| | | | Relative |
|---|---|---|---|
| Subsequence | N | Probability | Probability |
| PSP | 5 | 4.48 × 10⁻⁵ | — |
| HKP | 3 | 6.4 × 10⁻⁵ | — |
| HPR | 3 | 0.000218 | — |
| CCY | 1 | 0.000344 | 224.2562 |
| SNT | 3 | 0.000415 | — |
| FVN | 2 | 0.000524 | — |
| LLR | 3 | 0.000631 | — |
| RLL | 3 | 0.000631 | — |
| TCC | 1 | 0.000731 | 105.5608 |
| ISG | 2 | 0.000771 | — |
| GQA | 2 | 0.000776 | — |
| DHR | 2 | 0.000833 | — |
| PHS | 3 | 0.000907 | — |
| YSD | 2 | 0.000959 | — |
| PIK | 2 | 0.001057 | — |
| LGQ | 2 | 0.001334 | — |
| ASW | 2 | 0.00136 | — |
| APW | 2 | 0.001643 | — |
| KPN | 2 | 0.001693 | — |
| ARN | 2 | 0.001719 | — |
| DHH | 2 | 0.00173 | — |
| HHK | 2 | 0.00173 | — |
| PLS | 3 | 0.001804 | — |
| YTV | 2 | 0.001819 | — |
| SRY | 2 | 0.00218 | — |
| AKP | 2 | 0.002475 | — |
| AET | 2 | 0.002687 | — |
| IQP | 2 | 0.002706 | — |
| CMK | 1 | 0.002766 | 27.91259 |
| VQS | 2 | 0.002785 | — |
| PWL | 2 | 0.002815 | — |
| HIS | 2 | 0.003006 | — |
| ICM | 1 | 0.003252 | 23.73381 |
| QNL | 2 | 0.003315 | — |
| LQR | 2 | 0.003422 | — |
| TLG | 2 | 0.003433 | — |
| SDL | 2 | 0.003506 | — |
| HIP | 2 | 0.003626 | — |
| IPH | 2 | 0.003626 | — |
| QSR | 2 | 0.003693 | — |
| TQN | 2 | 0.003962 | — |
| QTR | 2 | 0.004089 | — |
| VHT | 2 | 0.004131 | — |
| PLD | 2 | 0.004227 | — |
| LRA | 2 | 0.004291 | — |
| TPG | 2 | 0.004465 | — |
| HQL | 2 | 0.005154 | — |
| RIC | 1 | 0.00526 | 14.67413 |
| CYN | 1 | 0.005422 | 14.23722 |
| PLF | 2 | 0.005518 | — |
| PAR | 2 | 0.005576 | — |
| NHP | 2 | 0.005765 | — |
| LSV | 2 | 0.005952 | — |
| PPL | 3 | 0.006153 | — |
| SPI | 2 | 0.006239 | — |
| STY | 2 | 0.006274 | — |
| YSP | 2 | 0.006824 | — |
| LDC | 1 | 0.007026 | 10.98647 |
| DTC | 1 | 0.007698 | 10.02722 |
| SLR | 2 | 0.007863 | — |
| SLQ | 2 | 0.008837 | — |
| NSP | 2 | 0.009871 | — |
| PRS | 2 | 0.010183 | — |
| QTS | 2 | 0.010524 | — |
| QPL | 2 | 0.010617 | — |
| THH | 2 | 0.011142 | — |
| LRL | 2 | 0.01197 | — |
| FPP | 2 | 0.013779 | — |
| HPL | 2 | 0.014108 | — |
| TPH | 2 | 0.016762 | — |
| THP | 2 | 0.016762 | — |
| PPV | 2 | 0.017774 | — |
| PVP | 2 | 0.017774 | — |
| NTT | 2 | 0.018131 | — |
| ASS | 2 | 0.020148 | — |
| HSS | 2 | 0.021447 | — |
| LST | 2 | 0.021974 | — |
| RPP | 2 | 0.023277 | — |
| PLT | 2 | 0.026255 | — |
| TPS | 2 | 0.028211 | — |
| TSP | 2 | 0.028211 | — |
| SPT | 2 | 0.028211 | — |
| PPA | 2 | 0.032254 | — |
| APP | 2 | 0.032254 | — |
| SPS | 2 | 0.042699 | — |
| TLT | 2 | 0.042847 | — |
| TTP | 2 | 0.054515 | — |

### Example 3

### Assembly of Subsequences into Motifs

The purpose of this Example was to assemble the subsequences identified in Example 2 into hair-binding peptide motifs.

Inspection showed that in the subsequences identified in Example 2, the significant 5-mers were made from significant 3-mers and that the significant 4-mers were either made from 3-mers or were Orphans or, in one case, was a Sink. Consequently to build the candidate sequences, we used only the 3-mer subsequences from this data. We only considered the 3-mer subsequences given in Table 4, which had a relative probability greater than 10. The 3-mer subsequences were classified as Linkers, Orphans, Sinks and Sources by using a spreadsheet to determine, for each particular subsequence, if there were any matches between the first two amino acids of that subsequence and the last two amino acids of any of the other subsequences and if there were any matches between the last amino acids of that subsequence and the first two amino acids of any of the other subsequences. For example, for subsequence PSP there were subsequences that ended with PS, TPS and SPS, and 3 subsequences, SPI, SPS, and SPT, that started with SP, so PSP was classified as a Linker. The results from the classification are shown in Table 5. Orphans were eliminated from further consideration.

TABLE 5
Classification of Subsequences of Three Amino Acids

| Subsequence | Classification |
|---|---|
| PSP | Linker |
| HKP | Linker |
| HPR | Linker |
| CCY | Linker |
| SNT | Source |
| FVN | Orphan |
| LLR | Linker |
| RLL | Linker |
| TCC | Linker |
| ISG | Sink |
| GQA | Sink |
| DHR | Orphan |
| PHS | Linker |
| YSD | Source |
| PIK | Sink |
| LGQ | Linker |
| ASW | Orphan |
| APW | Source |
| KPN | Sink |
| ARN | Sink |
| DHH | Source |
| HHK | Linker |
| PLS | Linker |
| YTV | Orphan |
| SRY | Sink |
| AKP | Source |
| AET | Orphan |
| IQP | Source |
| CMK | Sink |
| VQS | Source |
| PWL | Sink |
| HIS | Source |
| ICM | Linker |
| QNL | Sink |
| LQR | Sink |
| TLG | Source |
| SDL | Sink |
| HIP | Source |
| IPH | Linker |
| QSR | Linker |
| TQN | Source |
| QTR | Orphan |
| VHT | Orphan |
| PLD | Linker |
| LRA | Sink |
| TPG | Sink |
| HQL | Orphan |
| RIC | Source |
| CYN | Sink |
| PLF | Sink |
| PAR | Linker |
| NHP | Source |
| LSV | Sink |
| PPL | Linker |
| SPI | Linker |
| STY | Sink |
| YSP | Source |
| LDC | Sink |
| DTC | Source |
| SLR | Source |
| SLQ | Source |
| NSP | Source |
| PRS | Sink |
| QTS | Source |
| QPL | Linker |
| THH | Source |
| LRL | Linker |
| FPP | Source |
| HPL | Linker |
| TPH | Linker |
| THP | Source |
| PPV | Linker |
| PVP | Sink |
| NTT | Linker |
| ASS | Orphan |
| HSS | Sink |
| LST | Linker |
| RPP | Source |
| PLT | Sink |
| TPS | Linker |
| TSP | Linker |
| SPT | Sink |
| PPA | Linker |
| APP | Source |
| SPS | Linker |
| TLT | Orphan |
| TTP | Linker |

A Source subsequence was selected at random as a starting point. The subsequences that had their first two amino acids match the last two amino acids of the starting subsequence were noted. A candidate sequence was formed by concatenating the amino acids of the matching subsequence starting with the third amino acid to the starting Source subsequence. If there was more than one other subsequence whose first two amino acids matched the last amino acids of the starting subsequence, one was selected at random to use to begin.

The candidate sequence was used in a manner similar to the starting Source subsequence. Specifically, other subsequences that had their first two amino acids match the last two amino acids of the candidate sequence were noted. The candidate sequence was extended by concatenating the amino acids of the matching subsequence, starting with the third amino acid, to the candidate sequence. If there was more than one other subsequence whose first two amino acids matched the last amino acids of the candidate subsequence, one was selected at random for use. This method was continued to extend the candidate sequence until the sequences reached a length of 12-mers or the matching process led to a Sink subsequence. Forty-three sequences, shown in Table 6, were generated in this manner. This is not an exhaustive list of the possible sequences because no attempt was made to exhaustively enumerate all the possible sequences that could be built for the identified subsequences. Some of the sequences were terminated at 12-mers even though longer sequences were possible.

TABLE 6
Generated Hair-Binding Peptide Motifs

| Amino Acid Sequence | SEQ ID NO: |
|---|---|
| HPRS | 81 |
| AKPN | 82 |
| TQNL | 83 |
| SLQR | 84 |
| APWL | 85 |
| QSRY | 86 |
| YSDL | 87 |
| HISG | 88 |
| TSPT | 89 |
| PLSTY | 90 |
| VQSRY | 91 |
| TLGQA | 92 |
| QTSPT | 93 |
| NSPIK | 94 |
| YSPIK | 95 |
| NHPRS | 96 |
| FPPVP | 97 |
| RPPLD | 98 |
| QPLSV | 99 |
| THPLT | 100 |
| FPPVP | 101 |
| QPLSV | 102 |
| IQPLT | 103 |
| IQPLF | 104 |
| DHHKPN | 105 |
| THHKPN | 106 |
| HIPHSS | 107 |
| SNTTPG | 108 |
| PPLSTY | 109 |
| QTSPIK | 110 |
| NSPSPT | 111 |
| TTPHSP | 112 |
| APPARN | 113 |
| SNTTPHSS | 114 |
| SNTTPSPI | 115 |
| SNTTPSPT | 116 |
| QTSPSPSP | 117 |
| SPSPSPSP | 118 |
| SNTTPSPSP | 119 |
| THPLSNTT (concatenated THPL | 120 |
| and SNTT) | |
| SPIKRPPLS (concatenated SPIK | 121 |
| and RPPLS) | |
| RLLRLLRLLRA | 122 |
| RLLRLLRLLRLL | 123 |

### Example 4

### Demonstration of Motif Binding

The purpose of this Example was to demonstrate the binding of ten of the hair-binding peptide motifs generated in Example 3 to hair using an ELISA assay.

Ten hair-binding peptide motifs from Table 6 were selected for testing of their hair-binding activity. The ten peptides were synthesized by SynPep (Dublin, Calif.). As a positive control, a peptide that was identified as a hair-binding peptide having a high affinity for hair by Huang et al., supra, was used. The control peptide had the sequence TPPELLHGDPRS, given as SEQ ID NO:124. The peptides were biotinylated by adding a biotinylated lysine residue at the C-terminus of the amino acid binding sequences for detection purposes and an amidated cysteine was added to the C-terminus of the sequence.

Bleached hair samples were prepared and placed into wells of a custom 24-well biopanning apparatus, as described in Example 1. The hair was blocked with blocking buffer (SuperBlock™ from Pierce Biotechnology, Inc., Rockford, Ill.) at room temperature for 1 h, followed by six washes with TBST-0.5%, 2 min each, at room temperature. Various concentrations of biotinylated, binding peptide were added to each well, incubated for 15 min at 37° C., and washed six times with TBST-0.5%, 2 min each, at room temperature. Then, streptavidin-horseradish peroxidase (HRP) conjugate (Pierce Biotechnology, Inc.) was added to each well (1.0 μg per well), and incubated for 1 h at room temperature. After the incubation, the conjugate solution was removed and the wells were washed six times with TBST-0.5%, 2 min each, at room temperature. TMB substrate (200 μL) (Pierce Biotechnology, Inc.) was added to each well and the color was allowed to develop for between 5 to 30 min, typically for 10 min, at room temperature. Then, stop solution (200 μL of 2 M H₂SO₄) was added to each well and the solutions were transferred to a 96-well plate and the A₄₅₀ was measured using a microplate spectrophotometer (Molecular Devices, Sunnyvale, Calif.). The resulting absorbance values, were used to calculate the binding activity of each hair-binding peptide motif relative to the positive control sequence. The results are presented in Table 7.

TABLE 7
Binding Activities of Selected Hair-Binding Peptide Motifs

| | Binding Activity |
|---|---|
| SEQ ID NO: | % Relative to Control |
| 90 | 53.0 |
| 92 | 86.6 |
| 95 | 1.9 |
| 97 | 81.5 |
| 98 | 90.3 |
| 99 | 84.6 |
| 119 | 94.5 |
| 120 | 88.3 |
| 121 | 4.4 |
| 123 | 127.1 |
| 124 | 100.0 |

As can be seen from the results in the table, several peptides, specifically, SEQ ID NOs:98, 119, and 123, exhibited a binding activity comparable to or greater than that of the positive control peptide, which is a very strong hair-binder. Most of the other peptides showed significant binding to hair, but had less activity than the control. Only two of the peptides, specifically SEQ ID NOs: 95 and 121, had low binding activity to hair compared to the control. These results demonstrate that the method of the invention is useful in generating peptide motifs having a high binding affinity for bleached hair.

What is claimed is:

1. A method for non-empirically generating a sequence of a peptide motif having binding affinity for a substrate comprising the steps of:
a) providing a first population of substrate-binding peptides, each having a known amino acid sequence;
b) identifying all subsequences comprising at least two amino acids contained within the population of substrate-binding peptides of (a);
c) selecting those subsequences of (b) that occur statistically more frequently than by random chance to produce a statistically significant population of subsequences;
d) identifying multiples of statistically significant subsequences that have at least two amino acid patterns in common; and
e) assembling the multiples of statistically significant subsequences of (d) to generate at least one new peptide motif having binding affinity for a substrate, wherein said new peptide motif is not contained within the first population of substrate-binding peptides.

2. A method according to claim 1 wherein the substrate is selected from the group consisting of body surfaces, pigments, print media, carbon nanotubes, semiconductors, and polymers.

3. A method according to claim 2 wherein the body surfaces are selected from the group consisting of hair, skin, nails, teeth,

4. A method according to claim 1 wherein after step (e) the at least one new peptide motif having binding affinity for a substrate is further screened for substrate binding activity.

5. A method according to claim 1 wherein the population of substrate-binding peptides is combinatorially generated.

6. A method according to claim 5 wherein the combinatorial method of generation the population of substrate-binding peptides is selected from the group consisting of phage display, bacterial display, yeast display, and combinatorial solid phase peptide synthesis.

7. A method according to claim 1 wherein the population of substrate-binding peptides consists of at least about 50 unique peptides.

8. A method according to claim 1 wherein the population of substrate-binding peptides consists of at least about 75 unique peptides.

9. A method according to claim 1 wherein the population of substrate-binding peptides consists of at least about 100 unique peptides.

10. A method according to claim 1 wherein the subsequences of (c) occur statistically at least about five times more frequently than by random chance.

11. A method according to claim 1 wherein the subsequences of (c) occur statistically at least about ten times more frequently than by random chance.

12. A method according to claim 1 wherein the subsequences of (c) occur statistically at least about twenty times more frequently than by random chance.

13. A method according to claim 1 wherein the subsequences of step (b) are two to about five amino acids in length.

14. A method according to claim 1 wherein the at least one new peptide motif of step (e) is 3 to about 50 amino acids in length.

15. A hair care composition comprising a peptide motif that binds to hair generated by the process of claim 1.

16. A hair care composition according to claim 15 wherein the composition is a colorant.

17. A hair care composition according to claim 15 wherein the composition is a shampoo.

18. A skin care composition comprising a peptide motif that binds to skin generated by the process of claim 1.

19. A nail care composition comprising a peptide motif that binds to nails generated by the process of claim 1.

20. A tooth care composition comprising a peptide motif that binds to teeth generated by the process of claim 1.

21. A peptide motif having binding affinity for hair selected from the group consisting of: SEQ ID NOs:81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 20, 121, 122, and 123.

22. A hair binding composition comprising a peptide motif having binding affinity for hair selected from the group consisting of: SEQ ID NOs: 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 20, 121, 122, and 123.

23. A method for modifying hair comprising:
a) providing a hair binding peptide motif generated according to the method of claim 1;
b) contacting the hair binding peptide motif of (a) with a hair conditioning agent to generate a hair care composition; and
c) applying the hair binding composition of (b) to hair for a period of time sufficient to cause the hair to be modified.

24. A method according to claim 23 wherein the hair binding motif comprises the amino acid sequence selected from the group consisting of SEQ ID NOs: 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 20, 121, 122, and 123.


SEQUENCE LISTING (128 sequences)

SEQ ID NO: 1 - protein, 12 aa, Artificial Sequence
  Member of a population of hair-binding peptides
AETVESDLAKSH

SEQ ID NO: 2 - protein, 12 aa, Artificial Sequence
  Member of a population of hair-binding peptides
AKPISQHLQRGS

SEQ ID NO: 3 - protein, 12 aa, Artificial Sequence
  Member of a population of hair-binding peptides
ALKQDNTILLRE

SEQ ID NO: 4 - protein, 12 aa, Artificial Sequence
  Member of a population of hair-binding peptides
ANLQRMTPSSLL

SEQ ID NO: 5 - protein, 12 aa, Artificial Sequence
  Member of a population of hair-binding peptides
ANVQSHVDFQTR

SEQ ID NO: 6 - protein, 12 aa, Artificial Sequence
  Member of a population of hair-binding peptides
ASQTQNVRHSWP

SEQ ID NO: 7 - protein, 12 aa, Artificial Sequence
  Member of a population of hair-binding peptides
ASSDHHIPHSST

SEQ ID NO: 8 - protein, 12 aa, Artificial Sequence
  Member of a population of hair-binding peptides
AYFPYPLSTYRF

SEQ ID NO: 9 - protein, 12 aa, Artificial Sequence
  Member of a population of hair-binding peptides
DDFAKPYFSDTR

SEQ ID NO: 10 - protein, 12 aa, Artificial Sequence
  Member of a population of hair-binding peptides
DHHKSNTLGQAS

SEQ ID NO: 11 - protein, 12 aa, Artificial Sequence
  Member of a population of hair-binding peptides
DHRICMKTSPPL

SEQ ID NO: 12 - protein, 12 aa, Artificial Sequence
  Member of a population of hair-binding peptides
DPRSTHLFVQSG

SEQ ID NO: 13 - protein, 12 aa, Artificial Sequence
  Member of a population of hair-binding peptides
DSTYKVSNRSLQ

SEQ ID NO: 14 - protein, 12 aa, Artificial Sequence
  Member of a population of hair-binding peptides
DSYDSNMFPPYI

SEQ ID NO: 15 - protein, 12 aa, Artificial Sequence
  Member of a population of hair-binding peptides
EQISGSLVAAPW

SEQ ID NO: 16 - protein, 12 aa, Artificial Sequence
  Member of a population of hair-binding peptides
ESQSRQESLQIA

SEQ ID NO: 17 - protein, 12 aa, Artificial Sequence
  Member of a population of hair-binding peptides
FASGEHHTSPMD

SEQ ID NO: 18 - protein, 12 aa, Artificial Sequence
  Member of a population of hair-binding peptides
FSFENFLSDRSH

SEQ ID NO: 19 - protein, 12 aa, Artificial Sequence
  Member of a population of hair-binding peptides
GKAFVNQVRSSA

SEQ ID NO: 20 - protein, 12 aa, Artificial Sequence
  Member of a population of hair-binding peptides
GRRLLLRLTPGG

SEQ ID NO: 21 - protein, 12 aa, Artificial Sequence
  Member of a population of hair-binding peptides
GYSPIKRPPLDC

SEQ ID NO: 22 - protein, 12 aa, Artificial Sequence
  Member of a population of hair-binding peptides
HHSSRYSDVLAV

SEQ ID NO: 23 - protein, 12 aa, Artificial Sequence
  Member of a population of hair-binding peptides
HISPGWSPHRSD

SEQ ID NO: 24 - protein, 12 aa, Artificial Sequence
  Member of a population of hair-binding peptides
HNQSRYYTGKLH

SEQ ID NO: 25 - protein, 12 aa, Artificial Sequence
  Member of a population of hair-binding peptides
HQLSVRDWPLST

SEQ ID NO: 26 - protein, 12 aa, Artificial Sequence
  Member of a population of hair-binding peptides
HRQTSLPSPIAR

SEQ ID NO: 27 - protein, 12 aa, Artificial Sequence
  Member of a population of hair-binding peptides
HTPKNLSAPLTH

SEQ ID NO: 28 - protein, 12 aa, Artificial Sequence
  Member of a population of hair-binding peptides
IHKPNLRATPFS

SEQ ID NO: 29 - protein, 12 aa, Artificial Sequence
  Member of a population of hair-binding peptides
ITNSPSMHWSTF

SEQ ID NO: 30 - protein, 12 aa, Artificial Sequence
  Member of a population of hair-binding peptides
IVHQLQTRPIKP

SEQ ID NO: 31 - protein, 12 aa, Artificial Sequence
  Member of a population of hair-binding peptides
KIVNTYNRLQNL

SEQ ID NO: 32 - protein, 12 aa, Artificial Sequence
  Member of a population of hair-binding peptides
KLKHNHIPDPYL

SEQ ID NO: 33 - protein, 12 aa, Artificial Sequence
  Member of a population of hair-binding peptides
KNVDQSLRSFIV

SEQ ID NO: 34 - protein, 12 aa, Artificial Sequence
  Member of a population of hair-binding peptides
KQVEHVTTRTLT

SEQ ID NO: 35 - protein, 12 aa, Artificial Sequence
  Member of a population of hair-binding peptides
LDTSFPPVPFHA

SEQ ID NO: 36 - protein, 12 aa, Artificial Sequence
  Member of a population of hair-binding peptides
LGHTTGVNIYSP

SEQ ID NO: 37 - protein, 12 aa, Artificial Sequence
  Member of a population of hair-binding peptides
LMPPPWLGIASW

SEQ ID NO: 38 - protein, 12 aa, Artificial Sequence
  Member of a population of hair-binding peptides
LPKTTNPLLRAH

SEQ ID NO: 39 - protein, 12 aa, Artificial Sequence
  Member of a population of hair-binding peptides
LPLFPRELSVFT

SEQ ID NO: 40 - protein, 12 aa, Artificial Sequence
  Member of a population of hair-binding peptides
LPVRNMLQERWP

SEQ ID NO: 41 - protein, 12 aa, Artificial Sequence
  Member of a population of hair-binding peptides
NEVPARNAPWLV

SEQ ID NO: 42 - protein, 12 aa, Artificial Sequence
  Member of a population of hair-binding peptides
NITTPTFKSIPM

SEQ ID NO: 43 - protein, 12 aa, Artificial Sequence
  Member of a population of hair-binding peptides
NPPHPLALQQLR

SEQ ID NO: 44 - protein, 12 aa, Artificial Sequence
  Member of a population of hair-binding peptides
QLIPHAHVRPPA

SEQ ID NO: 45 - protein, 12 aa, Artificial Sequence
  Member of a population of hair-binding peptides
QSDYSGRLLGLG

SEQ ID NO: 46 - protein, 12 aa, Artificial Sequence
  Member of a population of hair-binding peptides
SDLPGLANSPAH

SEQ ID NO: 47 - protein, 12 aa, Artificial Sequence
  Member of a population of hair-binding peptides
SHISTSGPSPFG

SEQ ID NO: 48 - protein, 12 aa, Artificial Sequence
  Member of a population of hair-binding peptides
SKWLSHYSDMLI

SEQ ID NO: 49 - protein, 12 aa, Artificial Sequence
  Member of a population of hair-binding peptides
SLAPPVFMKFLK

SEQ ID NO: 50 - protein, 12 aa, Artificial Sequence
  Member of a population of hair-binding peptides
SLNWVTIPGPKI

SEQ ID NO: 51 - protein, 12 aa, Artificial Sequence
  Member of a population of hair-binding peptides
SMAHDPMAVRVY

SEQ ID NO: 52 - protein, 12 aa, Artificial Sequence
  Member of a population of hair-binding peptides
SNAHPLTRVLLA

SEQ ID NO: 53 - protein, 12 aa, Artificial Sequence
  Member of a population of hair-binding peptides
SNIQPQGTHWKT

SEQ ID NO: 54 - protein, 12 aa, Artificial Sequence
  Member of a population of hair-binding peptides
SNTTPSPTPHKP

SEQ ID NO: 55 - protein, 12 aa, Artificial Sequence
  Member of a population of hair-binding peptides
SPNPVTQNLIHT

SEQ ID NO: 56 - protein, 12 aa, Artificial Sequence
  Member of a population of hair-binding peptides
SSYEFDMSAVEP

SEQ ID NO: 57 - protein, 12 aa, Artificial Sequence
  Member of a population of hair-binding peptides
TAKWISGIDAPP

SEQ ID NO: 58 - protein, 12 aa, Artificial Sequence
  Member of a population of hair-binding peptides
THHKTPLHHHRT

SEQ ID NO: 59 - protein, 12 aa, Artificial Sequence
  Member of a population of hair-binding peptides
THPRSNTTASSG

SEQ ID NO: 60 - protein, 12 aa, Artificial Sequence
  Member of a population of hair-binding peptides
TLTSVTVRQPLF

SEQ ID NO: 61 - protein, 12 aa, Artificial Sequence
  Member of a population of hair-binding peptides
TLVIQPSLRLAS

SEQ ID NO: 62 - protein, 12 aa, Artificial Sequence
  Member of a population of hair-binding peptides
TPHSEKTVVLNS

SEQ ID NO: 63 - protein, 12 aa, Artificial Sequence
  Member of a population of hair-binding peptides
TPYWQTSTGTPE

SEQ ID NO: 64 - protein, 12 aa, Artificial Sequence
  Member of a population of hair-binding peptides
TQDSAQKSPSPL

SEQ ID NO: 65 - protein, 12 aa, Artificial Sequence
  Member of a population of hair-binding peptides
TQVPSPTHPAAF

SEQ ID NO: 66 - protein, 12 aa, Artificial Sequence
  Member of a population of hair-binding peptides
TYTKAATETFEL

SEQ ID NO: 67 - protein, 12 aa, Artificial Sequence
  Member of a population of hair-binding peptides
VHKPNIPPARNT

SEQ ID NO: 68 - protein, 12 aa, Artificial Sequence
  Member of a population of hair-binding peptides
VKPPLDPIHASW

SEQ ID NO: 69 - protein, 12 aa, Artificial Sequence
  Member of a population of hair-binding peptides
VPPSQPKQPNAL

SEQ ID NO: 70 - protein, 12 aa, Artificial Sequence
  Member of a population of hair-binding peptides
VSVKMPYNYVAY

SEQ ID NO: 71 - protein, 12 aa, Artificial Sequence
  Member of a population of hair-binding peptides
VVHTHATLGQAT

SEQ ID NO: 72 - protein, 12 aa, Artificial Sequence
  Member of a population of hair-binding peptides
WDTCCYNNHPMP

SEQ ID NO: 73 - protein, 12 aa, Artificial Sequence
  Member of a population of hair-binding peptides
WHAQFTPQPLSQ

SEQ ID NO: 74 - protein, 12 aa, Artificial Sequence
  Member of a population of hair-binding peptides
WSDSGLNHPRMR

SEQ ID NO: 75 - protein, 12 aa, Artificial Sequence
  Member of a population of hair-binding peptides
YNDFVNGHNPRT

SEQ ID NO: 76 - protein, 12 aa, Artificial Sequence
  Member of a population of hair-binding peptides
YPVPYQTHHMVQ

SEQ ID NO: 77 - protein, 12 aa, Artificial Sequence
  Member of a population of hair-binding peptides
YSQIPFAGPYTV

SEQ ID NO: 78 - protein, 12 aa, Artificial Sequence
  Member of a population of hair-binding peptides
YTHDHRLHPRLL

SEQ ID NO: 79 - protein, 12 aa, Artificial Sequence
  Member of a population of hair-binding peptides
YTTVNDAETPGH

SEQ ID NO: 80 - protein, 12 aa, Artificial Sequence
  Member of a population of hair-binding peptides
YTVHTVDPHSHQ

SEQ ID NO: 81 - protein, 4 aa, Artificial Sequence
  Hair-binding peptide motif
HPRS

SEQ ID NO: 82 - protein, 4 aa, Artificial Sequence
  Hair-binding peptide motif
AKPN

SEQ ID NO: 83 - protein, 4 aa, Artificial Sequence
  Hair-binding peptide motif
TQNL

SEQ ID NO: 84 - protein, 4 aa, Artificial Sequence
  Hair-binding peptide motif
SLQR

SEQ ID NO: 85 - protein, 4 aa, Artificial Sequence
  Hair-binding peptide motif
APWL

SEQ ID NO: 86 - protein, 4 aa, Artificial Sequence
  Hair-binding peptide motif
QSRY

SEQ ID NO: 87 - protein, 4 aa, Artificial Sequence
  Hair-binding peptide motif
YSDL

SEQ ID NO: 88 - protein, 4 aa, Artificial Sequence
  Hair-binding peptide motif
HISG

SEQ ID NO: 89 - protein, 4 aa, Artificial Sequence
  Hair-binding peptide motif
TSPT

SEQ ID NO: 90 - protein, 5 aa, Artificial Sequence
  Hair-binding peptide motif
PLSTY

SEQ ID NO: 91 - protein, 5 aa, Artificial Sequence
  Hair-binding peptide motif
VQSRY

SEQ ID NO: 92 - protein, 5 aa, Artificial Sequence
  Hair-binding peptide motif
TLGQA

SEQ ID NO: 93 - protein, 5 aa, Artificial Sequence
  Hair-binding peptide motif
QTSPT

SEQ ID NO: 94 - protein, 5 aa, Artificial Sequence
  Hair-binding peptide motif
NSPIK

SEQ ID NO: 95 - protein, 5 aa, Artificial Sequence
  Hair-binding peptide motif
YSPIK

SEQ ID NO: 96 - protein, 5 aa, Artificial Sequence
  Hair-binding peptide motif
NHPRS

SEQ ID NO: 97 - protein, 5 aa, Artificial Sequence
  Hair-binding peptide motif
FPPVP

SEQ ID NO: 98 - protein, 5 aa, Artificial Sequence
  Hair-binding peptide motif
RPPLD

SEQ ID NO: 99 - protein, 5 aa, Artificial Sequence
  Hair-binding peptide motif
QPLSV

SEQ ID NO: 100 - protein, 5 aa, Artificial Sequence
  Hair-binding peptide motif
THPLT

SEQ ID NO: 101 - protein, 5 aa, Artificial Sequence
  Hair-binding peptide motif
FPPVP

SEQ ID NO: 102 - protein, 5 aa, Artificial Sequence
  Hair-binding peptide motif
QPLSV

SEQ ID NO: 103 - protein, 5 aa, Artificial Sequence
  Hair-binding peptide motif
IQPLT

SEQ ID NO: 104 - protein, 5 aa, Artificial Sequence
  Hair-binding peptide motif
IQPLF

SEQ ID NO: 105 - protein, 6 aa, Artificial Sequence
  Hair-binding peptide motif
DHHKPN

SEQ ID NO: 106 - protein, 6 aa, Artificial Sequence
  Hair-binding peptide motif
THHKPN

SEQ ID NO: 107 - protein, 6 aa, Artificial Sequence
  Hair-binding peptide motif
HIPHSS

SEQ ID NO: 108 - protein, 6 aa, Artificial Sequence
  Hair-binding peptide motif
SNTTPG

SEQ ID NO: 109 - protein, 6 aa, Artificial Sequence
  Hair-binding peptide motif
PPLSTY

SEQ ID NO: 110 - protein, 6 aa, Artificial Sequence
  Hair-binding peptide motif
QTSPIK

SEQ ID NO: 111 - protein, 6 aa, Artificial Sequence
  Hair-binding peptide motif
NSPSPT

SEQ ID NO: 112 - protein, 6 aa, Artificial Sequence
  Hair-binding peptide motif
TTPHSP

SEQ ID NO: 113 - protein, 6 aa, Artificial Sequence
  Hair-binding peptide motif
APPARN

SEQ ID NO: 114 - protein, 8 aa, Artificial Sequence
  Hair-binding peptide motif
SNTTPHSS

SEQ ID NO: 115 - protein, 8 aa, Artificial Sequence
  Hair-binding peptide motif
SNTTPSPI

SEQ ID NO: 116 - protein, 8 aa, Artificial Sequence
  Hair-binding peptide motif
SNTTPSPT

SEQ ID NO: 117 - protein, 8 aa, Artificial Sequence
  Hair-binding peptide motif
QTSPSPSP

SEQ ID NO: 118 - protein, 8 aa, Artificial Sequence
  Hair-binding peptide motif
SPSPSPSP

SEQ ID NO: 119 - protein, 9 aa, Artificial Sequence
  Hair-binding peptide motif
SNTTPSPSP

SEQ ID NO: 120 - protein, 8 aa, Artificial Sequence
  Hair-binding peptide motif
THPLSNTT

SEQ ID NO: 121 - protein, 9 aa, Artificial Sequence
  Hair-binding peptide motif
SPIKRPPLS

SEQ ID NO: 122 - protein, 11 aa, Artificial Sequence
  Hair-binding peptide motif
RLLRLLRLLRA

SEQ ID NO: 123 - protein, 12 aa, Artificial Sequence
  Hair-binding peptide motif
RLLRLLRLLRLL

SEQ ID NO: 124 - protein, 12 aa, Artificial Sequence
  Control hair-binding peptide
TPPELLHGDPRS

SEQ ID NO: 125 - protein, 8 aa, Artificial Sequence
  Amino acid sequence of the Caspase3 cleavage site
LESGDEVD

SEQ ID NO: 126 - DNA, 20 bp, Artificial Sequence
  Primer
ccctcatagttagcgtaacg

SEQ ID NO: 127 - protein, 4 aa, Artificial Sequence
  Amino acid sequence of reference subsequence
AHQR

SEQ ID NO: 128 - protein, 5 aa, Artificial Sequence
  Amino acid sequence of reference subsequence
AHQRN